(19) United States
(12) Patent Application Publication
(10) Pub. No.: US 2012/0060418 A1
(43) Pub. Date: Mar. 15, 2012

(54) CATALYTIC GASIFICATION OF ORGANIC MATTER IN SUPERCRITICAL WATER

(75) Inventors: Michael Epstein, Rishon LeZion (IL); Abraham Kribus, Rehovot (IL); Alexander Berman, Kiriat Ekron (IL)

(73) Assignees: Ramot At Tel-Aviv University Ltd., Tel-Aviv (IL); Yeda Research and Development Co. Ltd., Rehovot (IL)

(21) Appl. No.: 13/320,856

(22) PCT Filed: May 20, 2010
(86) PCT No.: PCT/IL2010/000403

§ 371 Date: Nov. 16, 2011

Related U.S. Application Data
(60) Provisional application No. 61/179,770, filed on May 20, 2009.
(60) Provisional application No. 61/252,754, filed on Oct. 19, 2009.

(51) Int. Cl. C10L 3/08; B01J 21/04; B01J 23/34; B01J 21/10; B01J 21/06; H02K 7/18; B01J 23/889; B01J 23/83; B01J 23/656; B01J 23/63; B01J 27/232; C07C 31/04; B01J 19/08; B01J 23/10

(52) U.S. Cl. 48/127.7; 422/162; 502/355; 502/324; 502/340; 502/349; 502/303; 502/328; 502/332; 502/335; 502/337; 502/325; 502/174; 502/330; 568/840; 290/1 R

(57) ABSTRACT

A catalyst system including at least one metal and an oxide support, said oxide support including at least one of Al₂O₃, MnₓO_{y}, MgO, ZrO₂, and La₂O₃, or any mixtures thereof; said catalyst being suitable for catalyzing at least one reaction under supercritical water conditions is disclosed. Additionally, a system for producing a high-pressure product gas under super-critical water conditions is provided. The system includes a pressure reactor accommodating a feed mixture of water and organic matter; a solar radiation concentrating system heating the pressure reactor and elevating the temperature and the pressure of the mixture to about the water critical temperature point and pressure point or higher. The reactor is configured and operable to enable a supercritical water process of the mixture to occur therein for conversion of the organic matter and producing a high-pressure product fuel gas.

### FIELD OF THE INVENTION

This invention relates to supercritical water gasification and in particular to catalytic gasification of organic matter in supercritical water.

### REFERENCES

The following references are considered to be pertinent for the purpose of understanding the background of the present invention:
1. Matsumura, Y. et al (2005) Biomass gasification in near- and super-critical water: Status and prospects Biomass Bioenergy 29, 269-292
2. Clifford, T. (1998) Fundamentals of Supercritical Fluids. Oxford University Press, New York.
3. Gasafi E., Meyer L., and Schebek L. (2007) Exergetic efficiency and options for improving sewage sludge gasification in supercritical water. Int. J. Energy Res. 31, 346-363
4. Sondreal E. A. et al (2001) Review of advances in combustion technology and biomass cofiring. Fuel Processing Tech. 71, 7-38
5. Hao X., Guo L., Zhang X, Guan Y. (2005) Hydrogen production from catalytic gasification of cellulose in supercritical water. Chemical Engineering J. 110, 57-65
6. Elliott D. C., Sealock L. J., Baker E. G. Jr. (1993) Ind. Eng. Chem. Res. 32, 1542-8
7. Yoshida, T., Oshima, Y., Matsumura, Y. (2004) Gasification of biomass model compounds and real biomass in supercritical water. Biomass & Bioenergy 26, 71-78
8. Osada M., Sato O., Arai K., Shirai M. (2006) Stability of Supported Ruthenium Catalysts for Lignin Gasification in Supercritical Water. Energy & Fuels 20, 2337-2343
9. Minowa T., Ogi, Z. F. (1998) Cellulose decomposition in hot-compressed water with alkali or nickel catalyst. J. Supercritical Fuels 13, 253-9
10. Berman A., Karn R. K., Epstein M. (2007) Steam reforming of methane on a Ru/Al2O3 catalyst promoted with Mn oxides for solar hydrogen productiont. Green Chem. 9, 626-31
11. Pitz-Paal R., et al (2007) Development steps for parabolic trough solar power technologies with maximum impact on cost reduction. J. Solar Energy Engineering 129, 371-377
12. Kolb G. J., Diver R. B., Siegel N. (2007) Central-station solar hydrogen power plant. J. Solar Energy Engineering 129, 179-183
13. Obara I, Tanaka Y, Magoshi R, Yokota H, Shige T, Takita K (1998) Design of 600 degrees C. class 1000 MW steam turbine. JSME Int. J. Series B 41, 734-739
14. Yoshida Y. et al (2003) Comprehensive comparison of efficiency and CO2 emissions between biomass energy conversion technologies—position of supercritical water gasification in biomass technologies. Biomass and Bioenergy 25, 257-272
15. Yogev A, Yakir D. (1999) Bioreactor and system for improved productivity of photosynthetic algae. U.S. Pat. No. 5,958,761.
16. Avron and Ben-Amotz (1992) Donaliella: Physiology, Biochemistry, and Biotechnology, CRC press.
17. Elliott D. C., Sealock I. J and Baker E. G., U.S. Pat. No. 5,616,154, Apr. 1, 1997.

### BACKGROUND OF THE INVENTION

Biofuels are fuels extracted from biomass, which is either specifically grown for fuel production (“energy crops”), or obtained as organic waste from other processes (e.g., municipal waste, wood industry residues, sewage, industrial sludge). Examples of biofuels based on energy crops include ethanol produced from corn or sugar cane, and biodiesel produced from jatropha seed oil. In production of biofuels from energy crops, typically less than half of the plant matter is converted into a usable biofuel, and the rest becomes a waste stream, still having high content of organic matter, which has not been used in the fuel extraction process.

Exploiting a larger fraction of the organic matter to produce “third generation biofuels” could become possible with the help of engineered micro-organisms that are capable of breaking down cellulose. However, such processes are still under development and their effectiveness is not yet proven.

Waste with high content of organic matter, obtained either from conventional biofuel production or from other sources, can be used to generate heat and electricity, by filtering, drying and combustion. However, these treatment steps are expensive and energy-intensive, and conversion efficiency to electricity in direct combustion of biomass residues is low.

Another known process to extract useful energy from biomass residues is catalytic gasification to produce a gaseous mix containing mostly hydrogen, carbon dioxide, and carbon monoxide (“syngas”). This however is known to be done at high temperatures of at least 800° C., requiring complex and expensive process equipment. Undesirable side effects in this process can be incomplete conversion and the production of charcoal residues.

### GENERAL DESCRIPTION

There is a need in the art for a process that can convert organic matter into a useful fuel, achieving full conversion with minimal remaining waste, while requiring operation conditions (temperature and pressure) that are easy to realize using conventional industrial means.

The present invention provides a novel technique of supercritical water gasification (SCWG). Generally, SCWG is a gasification process that can be performed at a much lower temperature relative to conventional gasification. The lower temperature enables the use of standard materials and industrial equipment, thus reducing the complexity and cost of the process. The SCWG process is performed at temperature and pressure above the critical point of water: 374° C. and 220-221 bars. The resulting product is a gas mixture composed of hydrogen, carbon dioxide, carbon monoxide and C₁-C₄ hydrocarbons. This product can be used as is, or further upgraded by separating the high-value fuel constituents such as hydrogen.

Generally, the working temperatures for thermo-chemical gasification of biomass and other organic materials, in particular organic waste, are usually set to be above 600° C., in order to maximize the organic matter conversion and to minimize the formation of tar and char. Performing the gasification under conditions near the critical point of water (374° C., 221 bar) offers an alternate path that can be done at much lower temperature.

By using the teachings of the present invention, thermo-chemical gasification of biomass and other organic materials, in particular organic waste, may be achieved for producing renewable fuels. Performing the gasification under conditions near the critical point of water enables inter alia to perform the gasification at lower temperature relative to conventional gasification, to use water as solvent and reactant in addition to its role as carrier fluid and to complete conversion of the organic matter to combustible gaseous fuel without formation of tars and other undesired products. The gasification process involves complex chemistry including reactions of pyrolysis, steam reforming, hydrogenation, methanation and water-gas shift. An idealized stoichiometric equation for the overall reaction between biomass (cellulose in the example here) and water to produce a mixture of hydrogen and carbon dioxide is:

C₆H₁₀O₅+7H₂O→6CO₂+12H₂ (1)

A more detailed description of the gasification process, for p-cresol (a model compound) may be:

Steam reforming C₇H₈O+6H₂O→7CO+10H₂

Methanation CO+3H₂→CH₄+H₂O

Hydrogenation C₇H₈O+H₂→C₇H₈+H₂O

Water-gas shift CO+H₂O→CO₂+H₂ (2)

SCWG produces a gas mixture rich in hydrogen, typically about 55% H₂, 5% CO, 35% CO₂ and 5% CH₄.

However, according to the conventional approach, as biomass does not react well with steam at low temperature condition, significant amounts of tar and char are formed.

The novel approach of the present invention is based on the use of a novel catalyst system suitable for catalyzing at least one reaction under supercritical water conditions. The reactions may be selected from supercritical water gasification and decomposition of organic compounds in aqueous phase.

The catalyst system of the present invention is operable under supercritical water conditions (i.e. for supercritical operation) and comprises at least one metal supported on an oxide support, the oxide support comprising at least one of the following oxides Al₂O₃, MnₓO_{y} (wherein x=1 to 3; y=1 to 4), MgO and ZrO₂ and La₂O₃ or any mixtures thereof. This catalyst system is very active and stable under SCWG conditions.

It should be understood that, in the present invention, a small amount of metal catalyst is stabilized by placing it on a specific support so that it remains stable in an oxidizing atmosphere. Generally, in a high temperature water environment, metal components tend to form oxides. The oxidized metals have little catalytic activity for either carbon gasification or methanation [17]. The rate of the metal (e.g. ruthenium) oxidation depends on amount of the active metal and support. The inventors of the present invention have found that by employing MnₓO_{y}, Al₂O₃, MgO and La₂O₃ or any mixtures thereof as a support, ruthenium is still in the active reduced form, so that the catalyst is operable and durable in an oxidizing atmosphere. More specifically, by using this stable catalyst system, in comparison with conventional metal catalyst supported by other supports, the rate of sintering of metal particles, coke and tar formation and substrate disintegration are significantly decreased.

Preferably, the metal is selected from ruthenium, rhodium and nickel or any mixtures thereof.

In some embodiments, the metal is ruthenium, and comprises at least about 98 weight percent of ruthenium. The catalyst system may comprise ruthenium in an amount in the range of about 1 to 5% weight percent.

In some embodiments, the catalyst system comprises about 1-2 weight percent of ruthenium.

In some embodiments, the catalyst system comprises alkali salts. The alkali salts comprise at least one of K₂CO₃, KOH, NaOH, Ca(OH)₂ and Na₂CO₃ or any mixtures thereof.

In some embodiments, the oxide support comprises MnₓO_{y} in a concentration not exceeding 10 wt. %, MgO in a concentration not exceeding 10 wt. %, La₂O₃ in a concentration not exceeding 10 wt. % and Al₂O₃ (e.g. α-alumina).

The SCWG is a process that can convert organic matter, such as low-quality organic residues, into high-quality gas or liquid fuel. Water near and above its critical point has unique features with significant benefits on the gasification process. Organic compounds have high solubility and complete miscibility in supercritical water. Transport properties in supercritical water (transfer of mass and heat) are increased relative to the processes in conventional gasification, since the water behaves as a single continuous dense phase. Water participates in the process as a solvent, reactant, and transport medium; this avoids the need for drying the source organic material.

There is also provided a method for providing a product gas, the method comprising: providing a reactant mixture containing water and organic matter; providing a catalyst system as defined above; reacting the reactant mixture in presence of the catalyst system under supercritical water conditions; thereby obtaining a product gas. The supercritical conditions comprise temperature of about 374° C. or higher and pressure about of 220 bar or higher.

The organic matter comprises low-quality organic residues and waste such as residues from fermentation, anaerobic digestion, biomass, organic waste with high moisture content, agricultural and forestry waste; bagasse or other organic waste from the food processing industry; waste from bioethanol or biodiesel production processes; organic sludge from water treatment plants and refineries, marine algae, algal biomass sludge, sewage sludge or algae broth.

In this connection, it should be understood that practically all organic material can be decomposed under the conditions of supercritical water gasification (SCWG), leaving as residues only small amounts of inorganic matter present in biomass. The organic matter comprises inter alia biomass polymers typically comprising cellulose, hemicellulose and lignin, the relative concentration of which can vary between plant species. In addition, the chemical structure of lignin in particular and hemicellulose are very complex molecules. The cellulose polymer is composed of repeating glucose units, and therefore glucose, which is a monomer of cellulose, can be used as a representative molecule. Subcritical water gasification of cellulose and glucose gives almost the same reaction products. In addition, hemicellulose is easily dissolved under subcritical water conditions in the range of 200-230° C. and 34.5 MPa and converts to approximately 90% monomeric sugar.

The product gas comprises high-quality gas containing hydrogen, carbon dioxide, methane and CO.

In some embodiments, the method comprises separating at least one of water, H₂ and CO₂ from the product gas and optionally sequestrating CO₂ from said product gas.

The method may also comprise processing the product gas to produce liquid fuel.

In some embodiments, the method comprises pumping the reactant mixture into a pressure reactor, the pressure of the mixture being elevated to about the water critical pressure point or higher; heating the pressure reactor to elevate the temperature of the pressurized mixture to about the water critical temperature point or higher; interacting between the reactant mixture and the catalyst system inside the pressure reactor to enable a super critical water gasification process to occur within the pressure reactor such that high pressure product gas is produced; powering a turbine with the product gas produced by the gasification process; and powering an electrical generator with the turbine.

The high-pressure product gas being a thermodynamically valuable stream, expanding this stream in a turbine before separation of the fuel enables to produce work, in addition to the fuel output, without affecting the fuel production chemistry.

The turbine may be a multi-stage (multi-expansion stage) supercritical turbine, having one or more expansion stages, and optionally the expanding high-pressure product gas may be re-heated between the turbine stages.

In other embodiments, a batch mode operation is applied and no pumping is used. The pressure is achieved only by heating the reactor. In this case, the reactor is filled with the feed mixture, closed and heated. After a certain lapse of time, depending on the specific feed mixture, the products are released. Therefore, the method comprises heating the pressure reactor to elevate the temperature and the pressure of the mixture to about the water critical temperature and pressure point or higher; interacting between the reactant mixture and the catalyst system inside the pressure reactor to enable a super critical water process to occur within the pressure reactor such that high pressure product gas is produced.

In some embodiments the method comprises powering a turbine with the product gas; and powering an electrical generator with the turbine.

In some embodiments, at least a part of the energy used for pre-heating the pressure reactor may be obtained by using the thermal energy of the high-pressure gas. In other words, the method comprises using thermal energy of the high-pressure product gas to elevate the temperature of the mixture accommodated in the pressure reactor (pre-heating).

In some embodiments, heating the pressure reactor comprises using a solar energy source.

The system of the present invention can be used with solar heating at around 400-500° C. Supercritical water gasification of organic matter can therefore enable for exploiting solar energy.

In other embodiments, the method comprises storing heat produced by the solar energy source to enable continuous gasification.

By using the SCWG system of the present invention complete conversion into combustible product gas containing hydrogen, carbon dioxide, methane and CO can be achieved. This product gas rich in hydrogen can be used to generate electricity in a fuel cell or by combustion as a fuel in a turbine or other engine. By using an appropriate post-process, liquid fuels such as methanol may be produced, having the advantage to be much easier to store and transport relatively to gaseous mixture. The high-pressure product gas may also be used for direct cogeneration of electrical power by expansion through a supercritical turbine. Negative greenhouse gas impact can be achieved by separation and sequestration of the CO₂. Moreover, SCWG can be used directly on raw biomass, such as algae, or for conversion of organic waste, such as residues from fermentation, anaerobic digestion or other biofuel production processes, sewage sludge and algae broth.

The present invention provides a SCWG system for converting from input organic matter (e.g. biomass and organic waste with high moisture content) into a product gas (e.g. high-quality renewable fuel), achieving full conversion with minimal remaining waste of renewable fuel (e.g. only the small amounts of inorganic matter present in the biomass), and optionally cogenerating electricity via a supercritical turbine (i.e. in addition to the fuel mixture). The SCWG system produces a high-pressure product gas under super-critical water conditions. When operating in a batch mode, the SCWG system of the present invention comprises a pressure reactor accommodating a feed mixture of water and organic matter; and a solar radiation concentrating system heating the pressure reactor and elevating the temperature and the pressure of the mixture to about the water critical temperature point and pressure point or higher. The reactor is thus configured and operable to enable a supercritical water process of the mixture to occur therein for conversion of the organic matter and for producing a high-pressure product fuel gas.

When operating in a continuous mode, the system comprises a pump for pumping the feed mixture into the pressure reactor, the pump being operable to elevate pressure of the mixture to be about the water critical pressure point or higher (e.g. slightly above).

The pressure reactor may be a solar reactor directly heated by the solar radiation concentrating system. Alternatively, the pressure reactor may be indirectly heated by the solar radiation concentrating system. The solar radiation concentrating system may comprise a solar collector directly elevating the temperature of the mixture to about 400° C. or higher (e.g. 500 or 600° C.). Alternatively, the solar radiation concentrating system may comprise a solar collector indirectly elevating the temperature of the mixture to temperatures about 400° C. or higher (e.g. 500 or 600° C.) by using a heat transfer fluid circuit. The solar collector may be selected from parabolic trough collector, solar tower, and solar tower comprising a tower reflector.

In some embodiments, the system comprises a turbine (e.g. steam turbine) connected to the pressure reactor for receiving the high pressure gas produced in the gasification process; and an electrical generator connected to the turbine, enabling cogeneration of electrical power.

The system may also comprise one or more separators for receiving the high-pressure gas (e.g. after expansion through the turbine) and separating at least one of water, H₂ and CO₂.

For example, the separator receiving the high-pressure gas and separating H₂ may be a Pressure Swing Adsorption (PSA) separator extracting H₂ from the mixture output stream. The PSA requires a certain input pressure, which may be provided by the last expansion stage in the turbine.

The water supercritical conditions are defined by a critical pressure point of 220 bars and higher and a critical temperature point of about 374° C. and higher.

In some embodiments, the system comprises a thermal storage unit configured and operable for continuously operating the pressure reactor regardless to the variations of solar energy.

In other embodiments, the system comprises a heat exchanger configured and operable to use the thermal energy of the high-pressure product gas to elevate the temperature of the mixture (pre-heat the mixture).

There is also provided a system for producing a high-pressure product gas under super-critical water conditions. When operating in a batch mode, the system comprises a pressure reactor accommodating a feed mixture of water and organic matter; a heating system heating the pressure reactor and elevating the temperature and the pressure of the feed mixture to about the water critical temperature point and pressure point or higher; the pressure reactor being configured and operable to enable a supercritical water gasification process of the mixture to occur therein for producing a high-pressure product gas; a turbine connected to the pressure reactor for receiving the gas produced in the gasification process; and an electrical generator connected to the turbine, enabling cogeneration of electrical power.

When operating in a continuous mode, the system comprises a pump for pumping the feed mixture into the pressure reactor, the pump being operable to elevate pressure of the mixture to be about the water critical pressure point or higher (e.g. slightly above).

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A-1B are schematic layouts of an example of the system of the present invention;

FIG. 2 represents an SCWG solar reactor indirectly heated by a solar radiation concentration system according to one embodiment of the present invention;

FIG. 3 represents the SCWG solar reactor of FIG. 2 heated by solar parabolic troughs;

FIG. 4 represents the SCWG solar reactor of FIG. 2 heated by a solar tower;

FIG. 5 represents a SCWG solar reactor directly heated by a solar tower reflector;

FIG. 6 represents a simulation of an example of the SCWG system of the present invention for simultaneous cogeneration of fuel and electricity;

FIG. 7 represents the overall efficiency of the system of the present invention at 600° C. for different feedstock mass fractions and different number of turbine expansion stages;

FIG. 8 represents the overall efficiency of the system of the present invention at fixed feedstock mass fraction for different temperatures and different number of turbine expansion stages; and;

FIGS. 9A-9B illustrates a schematic representation (9A) and experimental implementation (9B) of an example of the reactor of the present invention.

### DETAILED DESCRIPTION OF EMBODIMENTS

The present invention provides an appropriate catalyst system for converting organic matter at high efficiency and in a reasonably short time. It should be understood that under SCWG conditions, in long-term operation of the known systems of the kind specified, effectiveness of the catalyst decreases because of the oxidation of the metal components in the process environment, sintering of the metal particles, coke and tar formation, and substrate disintegration.

Alkali catalysts, for example sodium carbonate, have been employed [9] for increasing the gasification efficiency of cellulose. Other alkalis such as K₂CO₃, KOH, NaOH and Na₂CO₃ are known to catalyze the water-gas shift reaction with the formation of H₂ and CO₂ instead of CO. Other catalysts such as Pt and Pd having lower activity have also been tested. Tests were also conducted with copper, molybdenum, tungsten, chromium and zinc metals, but these also showed very low level of catalytic activity. Activity of oxide catalysts like CeO2, (CeZr)xO2 or CaO—MnO—Ce O2 is also low [5].

As mentioned above, to be stable under supercritical water conditions, the metal has to be supported by an appropriate support. Typically, the conventional catalyst supports are severely degraded in this reaction media [6]. For example, silica dissolves in high temperature water, and most of the forms of alumina react to form γ-AlOOH (boehmite) resulting in loss of physical integrity. As for the α-alumina, it might also not be stable because the SCWG operating conditions in the conventional techniques are actually outside the α-alumina range in the phase diagram. The monoclinic forms of zirconia, MgO, CeO₂ and Titania extrudate appear to be stable at these conditions [8].

The present invention provides an active catalyst system stable under supercritical water conditions supported and promoted with a suitable combination of oxides as substrate. The combination of oxides may be selected from MgO, Al₂O₃, ZrO₂, La₂O₃ and MnₓO_{y} oxides. The metal is selected from ruthenium, rhodium and nickel. The catalyst system can be monolithic, packed bed or fluidized and then filtered and separated later after the discharge of the gas products from the pressure reactor and recycled for further use. When the catalyst is monolithic, the catalyst and the substrate are stationary and the reactants flow through a stationary bed. When the catalyst is dispersed, the catalyst comprises particles that move with the reactants. At the exit of the pressure reactor, the particles are separated and recycled back to the inlet of the reactor.

Preferably, the catalyst system can be a ruthenium catalyst supported on α-alumina and promoted with Mn oxide for high temperature reforming of methane. Alternatively, the catalyst system may be a ruthenium catalyst supported on stabilized zirconia (monoclinic). The activity of the catalyst can be improved by addition of Mg or La oxides. The inventors have shown that some compositions of MgO, Al₂O₃ and Mn oxide are stable also under supercritical water conditions. This type of catalyst can be introduced into the SCWG reactor as part of the reactant mixture without need for a fixed catalyst bed. The catalyst is then expected to precipitate out of the products mixture and can be separated, collected, and recycled.

Another family of catalysts is dissolved in the water. Such catalyst system comprises soluble alkali catalysts such as K₂CO₃, KOH, NaOH, Ca(OH)₂ and Na₂CO₃. In particular, the inventors have found that the H₂ yield of SCWG of glucose with NaOH was almost 4 times higher than that without catalyst. Thus, a base catalyst has a positive effect to produce gaseous product such as H₂.

The addition of alkali salts increases the reaction rate and suppresses the formation of soot and tar. Experiments have shown that addition of KOH leads to a decrease in the CO concentration in the product gas. The addition of alkali salts, such as an acid-base catalyst, increases the rate of the water-gas shift reaction (CO+H₂ [symbol] CO₂+H₂). Therefore, SCWG with soluble alkali catalysts show substantial catalytic effect on the gasification of cellulose. This enables the possibility of using a reactor without a fixed catalytic bed, and resolves some of the operational problems that are associated with a fixed bed.

In particular, for glucose gasification in supercritical water, the inventors estimate that the major reaction pathways associated with hydrogen production include first the conversion of glucose to large amounts of water-soluble intermediates and most of these then contribute to produce CO. Some of the intermediates may be converted to CO₂ and H₂ by a steam reforming reaction (CₙHₘO_{y}+(2n−y) H₂O→nCO+(2n−y+m/2)H₂). The carbon monoxide formed may be finally converted to carbon dioxide and hydrogen through the water-gas shift reaction.

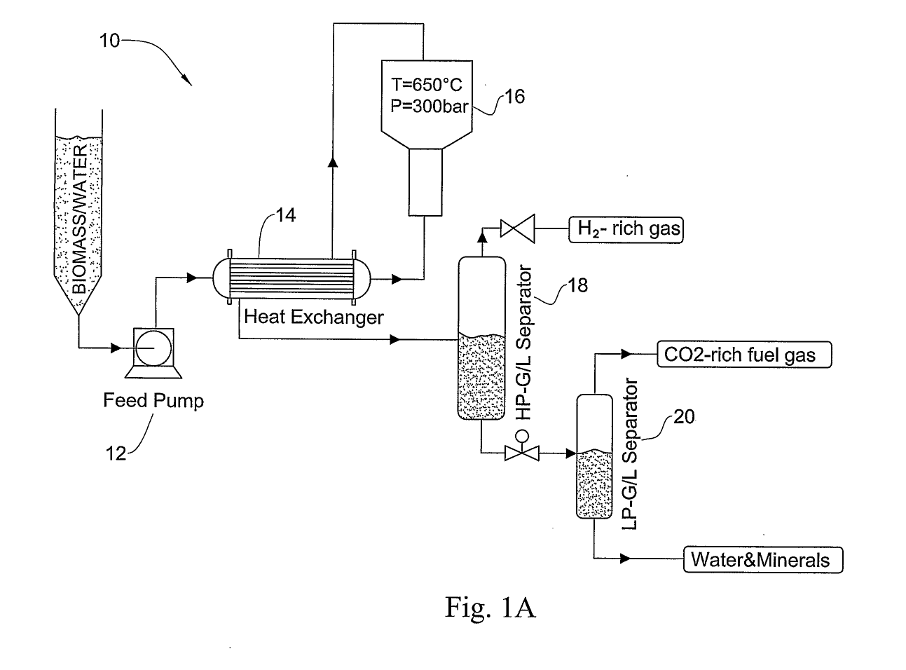

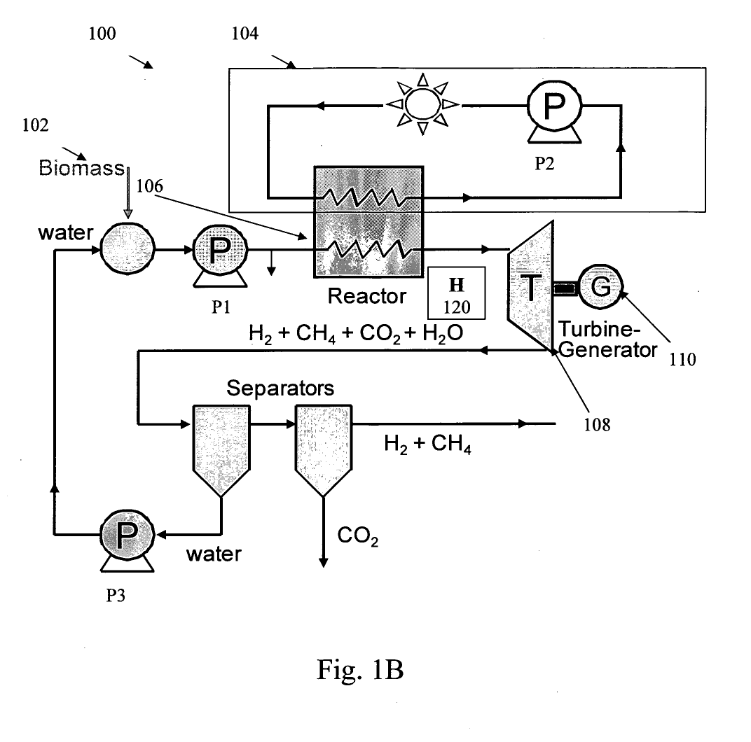

Another aspect of the present invention is providing a SCWG system for producing a high-pressure product gas under super-critical water conditions. In this connection, reference is made to FIG. 1A, illustrating a schematic layout of the SCWG system 100 of the present invention.

The mixture of organic matter and water is pressurized in a high-pressure pump 12 (e.g. feed pump) and enters the reactor 16 after preheating by using for example a heat exchanger 14 as illustrated in the figure. Supercritical water process of the mixture occurs in the pressure reactor 16 producing a high-pressure gas. The high-pressure product mixture is then separated, by a first separator 18 yielding one or more streams rich in a combination of hydrogen, CO and methane; and optionally by a second separator 20 separating a stream of CO2; and the remainder stream containing water and the inorganic residues originating from the biomass input. The water can be recovered and recycled back to the reactant side. Both hydrogen and methane can be used for producing heat and electricity. Carbon dioxide can be separated, captured and optionally sequestrated.

Reference is made to FIG. 1B, illustrating another schematic layout of the SCWG system 100 of the present invention. In this specific example, the system 100 comprises a pump P1 for pumping a reactant mixture containing water and organic matter (e.g. biomass 102). The pump P1 operates to elevate the pressure of the mixture above the water critical pressure thereof. Further provided in the system 100 is a solar radiation concentrating system 104 connected to a pressure reactor 106 associated with a pump P2 and operated for elevating the temperature of the mixture above the water critical temperature point. Supercritical water process of the mixture occurs in the pressure reactor 106 producing a high-pressure gas. A turbine 108 is connected to the pressure reactor 106 for receiving the gas produced in the gasification process, and an electrical generator 110 is in turn connected to the turbine 108 to thereby enabling cogeneration of electrical power.

After expanding through the turbine, the product gases are separated first from the water. The water can be recycled to the process via pump P3. Then, the CO₂ is separated by a conventional method and the final fuel gases can be used to further operate the turbine by their combustion or for other purposes like synthetic liquid fuel production.

It should be noted that a technique using partial contribution by heat recovery from the product gas and then burning of natural gas to provide the main heat source has been suggested [3]. It is also known that the heat source may be provided by combustion of a part of the biomass feed, or a part of the gas fuel output. A thermodynamic analysis of the SCWG process has shown that the main source of inefficiency, or energy loss, occurs during the heating of the water-biomass mixture by combustion [3].

In some embodiments, the present invention overcomes the above problem of inefficiency by appropriately heating the reactant water-biomass mixture using a solar energy source. Use of this source of energy has the advantage that no combustion is required and that the process is environmentally clean. It should be noted that the conventional annual average conversion efficiency from solar radiation to electricity in solar power plants, either parabolic trough or solar tower, is in the range of about 15-20%. The effective conversion efficiency from solar radiation to power, when going though the biomass conversion process, could be significantly higher than the efficiency of current solar power plants. The solar heat is primarily converted to chemical energy stored in the product fuel (assuming proper heat recovery in the process). By using the teachings of the present invention and by exploiting solar energy together with SCWG, conversion of chemical energy to electricity can be done very efficiently, for example in a fuel cell or a combined cycle power plant, leading to higher conversion efficiency relative to existing solar thermal power plants with conversion in a lower temperature turbine.

The concentrated solar energy (i.e. radiation) may be introduced into the pressure reactor in any of several conventional ways. The reactor may have an insulated cavity internally accommodating an arrangement of reactor tubes, the concentrated solar radiation being introduced into the reactor cavity and distributed to the internal reactor tubes.

The inventors have estimated that by using a solar concentrator field, the efficiency due to the field and solar receiver is 0.7, and then the system efficiency range is 0.36-0.48.

According to one embodiment of the present invention, the high-pressure product mixture is expanded through a supercritical (SC) turbine and generates electricity in addition to the production of fuel. Using supercritical steam cycles, the conversion efficiency from heat to electricity can reach more than 45%, compared to 35-40% for subcritical steam cycles. A SC steam turbine can accommodate the product gas mixture (with somewhat different physical properties instead of steam), while the range of temperature and pressure is similar to the SC steam cycle conditions.

Using a continuous SCWG process is effective because the heat from the product gas can be recovered and used to preheat the water/biomass mixture entering the solar reactor. Moreover, using the configuration of the system of the present invention, offers a new degree of freedom to design the gasification heating system and the solar reactor without constraints from heat recovery requirement.

In some embodiments, the thermal energy contained in the product gas can be used partly to generate electrical power in the turbine, and partly for preheating the incoming water-biomass mixture through a heat exchanger H 120 as illustrated in the FIG. 1B. In this specific example, the incoming feed mixture is therefore heated partly by the heat regeneration from the product gas and partly by an external heat input (solar energy or another heat source).

Additionally or alternatively, the heat from the compressed fluid leaving the turbine (in a cogeneration cycle) or the reactor (in the gasification-only cycle), is used for preheating the stream of reactants before it enters the reactor. This is done for example by using a preheat heat exchanger located before the reactor inlet (as illustrated for example in FIG. 1A). In general, not all the heat available in the exiting stream can be recovered, depending on the mixture composition, the heat exchanger characteristics, and the streams temperatures and pressures. The heat exchanger performance is defined by setting e.g. 5° C. approach between the streams in the heat exchanger. The remaining heat that was not recovered in the heat exchanger is extracted and disposed to the surroundings in the separator.

After expansion through the supercritical turbine, the temperature and the pressure of the mixture decrease. The mixture can then be separated and post-treated to generate the final gas or liquid fuel. The composition of the product gas, and its caloric value, do not change during the expansion. The cogeneration of electricity by expansion is therefore an additional energetic output of the process, increasing the overall conversion efficiency.

The operation conditions of the supercritical turbine, and in particular the mixture exit pressure and temperature, are selected to be compatible with subsequent post-processing steps. Several known processes (such as liquefaction, pressure swing absorption, chemical separation) can be used to separate the CO₂ from the product gas mixture, leaving a high quality fuel containing mostly hydrogen, CO and methane. The operation conditions of any heat recovery (e.g. heat exchanger) placed before the separator may be adjusted according to the chosen separation process. For example, a CO₂ separation process can be used, such as Pressure Swing Adsorption (PSA) [3], or water scrubber. The exit pressure from the supercritical turbine is chosen close to the inlet pressure required for the separator, for example around 2-7 bar for PSA. Separation of the CO₂ leads to higher quality fuel with a higher concentration of hydrogen. This fuel can be used in combustion to generate heat or to generate electricity in a power plant, or can be used to produce power in a fuel cell at high efficiency.

If CO₂ is separated, then an interesting option is its sequestration instead of release to the atmosphere, leading to reducing this greenhouse gas from the atmosphere. Since the CO₂ contains carbon from a renewable source, the sequestration actually has the effect of negative greenhouse gases emission which doubles the environmental impact of the process: not only replacing fossil fuel with a renewable fuel (zero net emission, neutral effect on the atmosphere), but actually a net reduction in atmospheric carbon.

Water vapor can be removed from the product gas mixture by cooling and condensation. Before separation of the water, the mixture can be subject to the water-shift reaction to convert the CO fraction of the product mixture to CO₂, thereby releasing an additional amount of hydrogen from decomposition of the water:

CO+H₂O→CO₂+H₂ (3)

This additional step can be performed either in a separate section of the gasification solar reactor, or in a separate reactor downstream of the gasification step.

The product gases from the SCW gasification process contain mainly H₂ and CO₂. Before or instead of separating the CO₂ and the hydrogen, CO and CO₂ can react and this mixture can be further processed catalytically in a known process to produce methanol: CO₂+3H₂→CH₃OH+H₂O. Methanol is a liquid fuel that can easily substitute current fossil-based liquid fuels in applications such as transportation, without the extensive modifications in infrastructure that would be necessary for substitution with a gaseous fuel.

Gasification of organic matter from biomass origin can apply to many types of feedstock, and provide the dual benefit of eliminating waste and generation of useful renewable energy. Possible feedstock materials can include agricultural and forestry waste; bagasse and other organic waste from the food processing industry; waste from bioethanol and biodiesel production processes; organic sludge from water treatment plants; etc.

Many types of organic feed materials can be used as input to the SCWG process. Raw biomass ('energy crops'), marine algae, which can be produced in high concentrations in solar bioreactors, or in ponds in marginal lands, can be used. Algae as feedstock for a gasification process may offer a significant advantage relative to other sources of biomass, since their composition is richer in simple carbohydrates that decompose easily, and therefore the requirements for catalysis are lower. Algae biomass can be selected and designed to provide high concentrations of carbohydrates that will be especially suitable and highly efficient in gasification reducing energy consumption and producing little waste.

Another class of biomass feedstock is the organic waste remaining after extraction of useful components of the original biomass. An attractive option is the waste from conventional biofuel extraction. In bio-ethanol production, for example, about half the original biomass remains as a wet solid waste. A similar fraction of waste remains in bio-diesel and biogas production. Using this wet organic waste to produce an additional fuel via SCWG raise the conversion rate of the biomass to nearly 100% of its organic content, making the maximum use of the raw material and avoiding the need to handle and dispose of the waste.

Other sources of feedstock can include the many types of organic waste that are generated in the food industry, in agricultural operations (animal waste, wood chips, stalks of food crops, etc.), and in many other industries. Municipal sewage may also be suitable, as is sludge from water treatment plants and refineries. Another very suitable source of organic waste is the algal biomass sludge remaining after extraction of commercially valuable compounds, e.g. β-carotene or glycerol, omega-3 etc.

The product gas mixture from the SCWG process can be used in several ways, to produce a range of fuels (gas and liquid), and to produce power. More specifically, the product gas mixture can be used directly as a fuel, essentially a substitute for natural gas (Synthetic Natural Gas (SNG)). The SNG can be combusted in a gas engine or a gas turbine (simple cycle or combined cycle) to produce electricity. The different gas composition may require some adjustment of the engine (e.g. compression ratio, ignition sequence). To minimize the impact of the different gas composition, the SNG may be mixed with natural gas in a ratio that would reduce the difference to a level that does not require any modification.

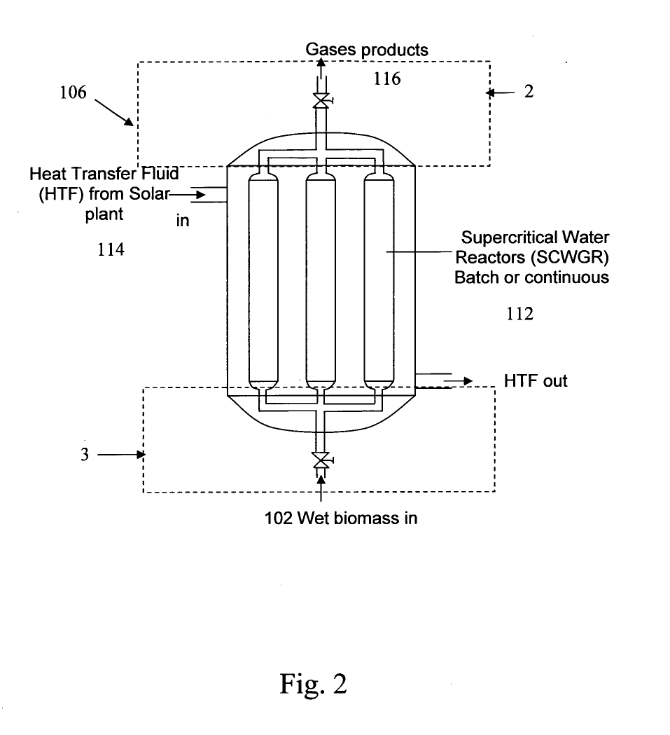

Reference is made to FIG. 2 illustrating a non-limiting example of the solar reactor 106 of the present invention, in which a feed mixture 102 containing water and organic matter (e.g. wet biomass) is pumped into the reactor 106. In this specific example, the solar reactor 106 comprises a plurality of vessels 112 to enable batch or continuous SCWG operation and the production of a high-pressure gas 116. The solar reactor 106 is heated by a heat transfer fluid (HTF) circuit 114 heated by a solar energy source. Reactions in accordance with the present invention may be conducted in continuous, batch, or semi-batch mode.

In case of continuous flow system operation, the wet biomass 102 can be introduced into the solar reactor 106 via an inlet valve by a pump at the bottom region 3 of the solar reactor. The top region 2 of the solar reactor 106 is fitted with an exhaust pipe for the withdrawal of the product synthesis gas 116.

In case of batch operation, the reactor may be filled with an appropriate amount of feed (i.e. organic matter) and the gas products are released when the reaction is completed.

The thermal energy required for the gasification process using a SCWG process is at temperature range of about 400° C. to 600° C. Heat at temperature of about 400° C. can be provided by solar collectors, for example of parabolic trough type [11]. It is also possible to perform the reaction at for example, around 500° C., and in this case, another solar collector technology, such as a solar tower, can be used [12]. The heat transport between the solar collector plant and the gasification solar reactor can be done by a suitable fluid, Heat transfer Fluid (HTF), such as an organic fluid, a molten salt, or a gas such as air.

Typically, the use of solar energy to produce electricity or a renewable fuel at high efficiency usually requires high temperature, for example in the range of 800-1200° C. Solar collectors operating at a moderate 400° C. are able to generate electricity, but the conversion efficiency is not high. Using the SCWG process enables to convert practically all of the collected solar energy at moderate temperature into a renewable fuel, which can be further used to generate electricity at high efficiency.

For example, parabolic trough collector can be used to effectively produce solar heat at temperatures around 400° C. The parabolic trough collectors may be used to heat the water-biomass slurry either directly or indirectly using an intermediate heat transfer fluid circuit.

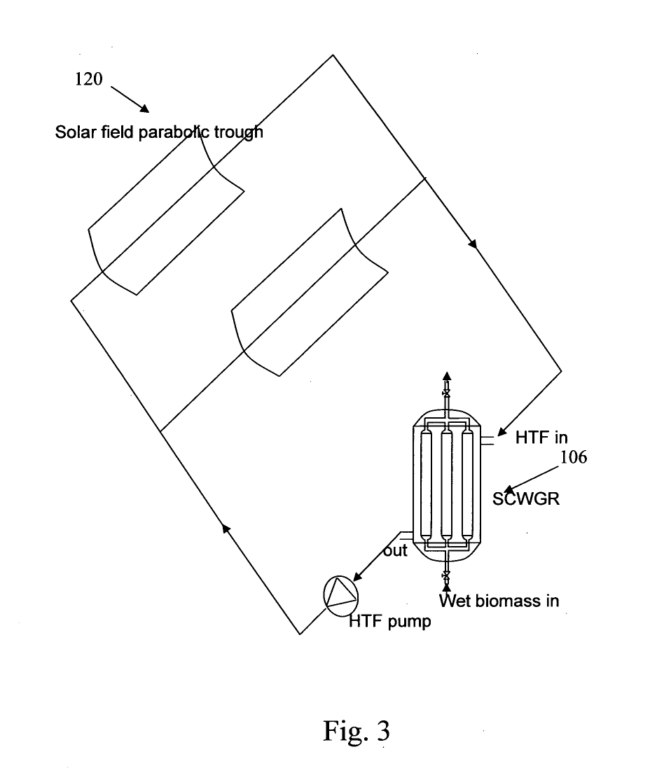

In this connection, reference is made to FIG. 3 representing the solar reactor 106 of FIG. 2 connected to a solar radiation concentrating system comprising a solar field parabolic trough 120.

The parabolic trough technology may even achieve higher temperatures than 400° C. by Direct Steam Generation (DSG) or with a molten salt heat transfer fluid. Considerably higher temperature, if needed, can be produced with a solar tower being another proven large-scale solar concentration technology.

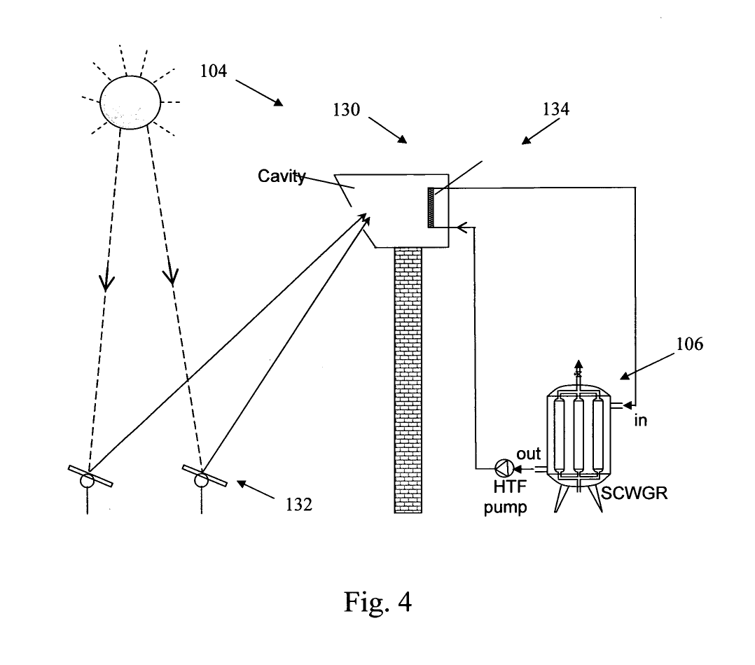

In this connection, reference is made to FIG. 4 representing the solar reactor 106 of FIG. 2 connected to a solar radiation concentrating system comprising a solar tower 130 and solar heliostats field 132.

The heliostats field 132 consists of a plurality of computer-controlled mirrors, which redirect solar radiation towards a solar heater/receiver 134 placed in the region of the focal points located on top of a central solar tower 130. The central solar tower 130 accommodates the solar heater 134, which heats the Heat Transfer Fluid (HTF). The HTF may be circulated by a pump to heat the SCWG reactor, which is placed on the ground.

Another solar radiation concentrating system, particularly useful in a large-scale solar energy plant may include a heliostats field, a tower reflector and a ground secondary concentrator associated with a solar receiver.

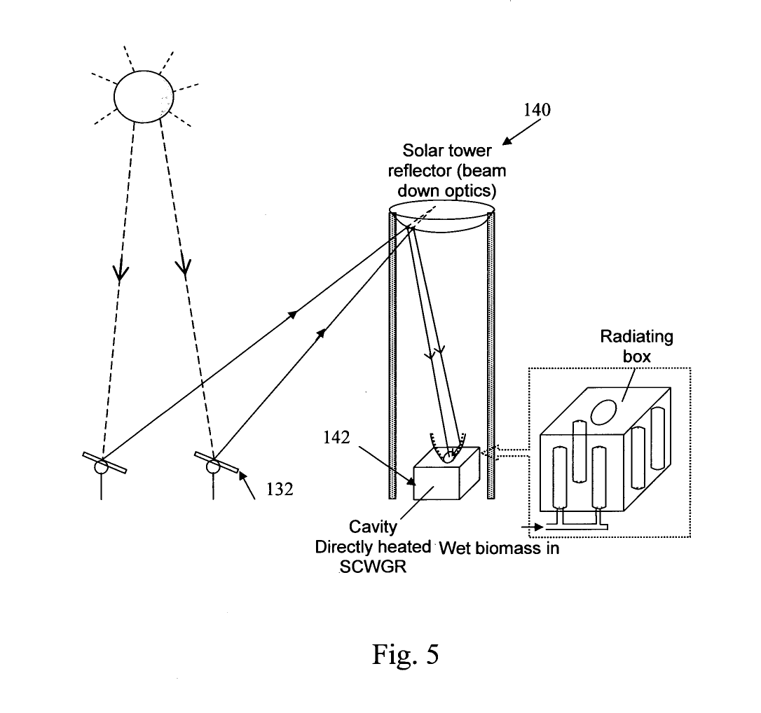

Reference is made to FIG. 5 representing the solar reactor 106 of FIG. 2 connected to a solar radiation concentrating system 104 comprising a solar tower reflector 140. As shown, the solar radiation concentrating system 104 comprises a primary concentrator in the form of a heliostats field 132 consisting of a plurality of concentrating mirrors mounted on the ground plane and a tower reflector 140. The incoming solar radiation is concentrated and reflected by the heliostats field 132 in the direction of the tower reflector 140 so as to be redirected thereby onto the ground solar cavity receiver 142 accommodating the solar gasification reactor 106.

The solar radiation concentrating system according to the present invention is not restricted to the characteristics thereof described above but rather may have any other appropriate design.

In some embodiments, the solar heat may be stored to enable continuous operation of the SCWG process, regardless of the variations in available sunlight. Advanced heat storage solutions at the relevant temperatures can also be used, for example by using composite phase change materials. A thermal storage unit can therefore be provided as a buffer between the solar collector and the gasification solar reactor. The thermal storage unit can use storage technologies such as a solid porous bed, a pure or composite phase change material bed, a dual storage tanks with hot and cold fluids, or a thermocline (e.g. with both hot and cold fluid) in a single storage tank.

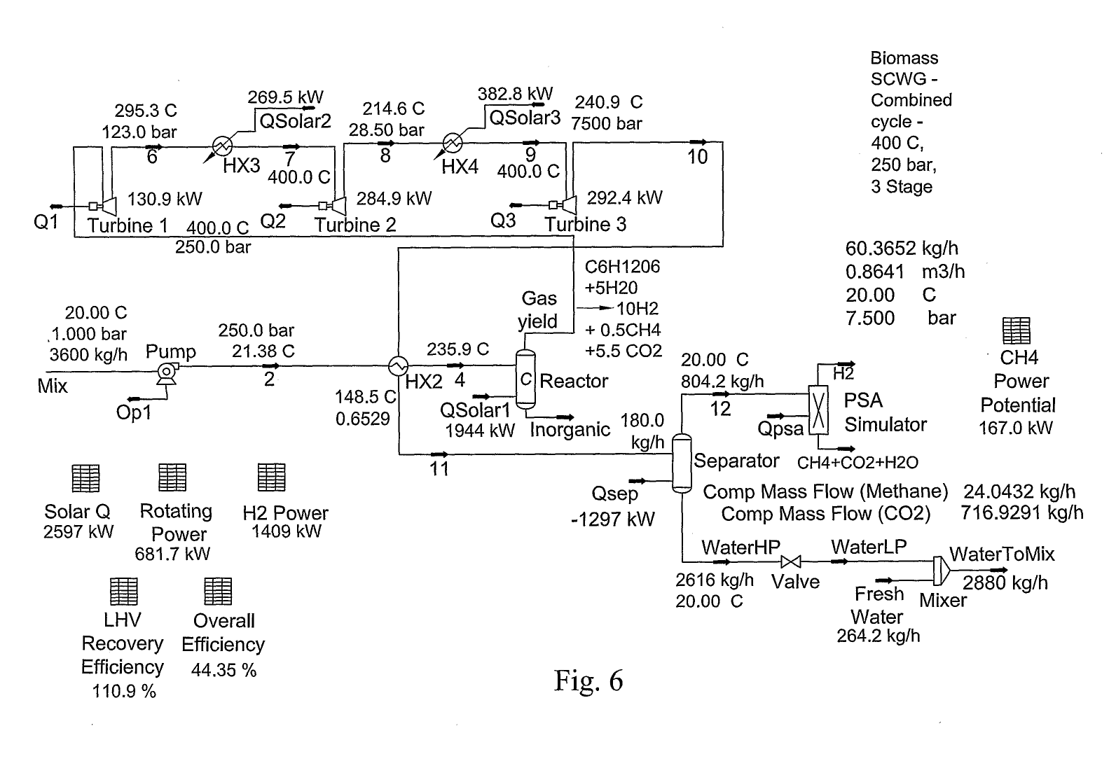

Reference is made to FIG. 6 illustrating an example of the SCWG system of the present invention for simultaneous cogeneration of fuel and electricity in one single cycle. In this specific and non-limiting example, the system comprises a SCWG reactor, a heat recovery heat exchanger that preheats the reactor, expansion valves to reduce the mixture pressure, and a series of separators to separate water, H₂ and methane. The methane is recycled to provide a part of the reactor heating. The rest of the heat is provided by an external source, which can be a conventional fuel such as natural gas, or solar energy from a solar concentrator field. The system also includes a turbine with 1-3 expansion stages and intermediate reheat heat exchangers, similar to a standard Rankine cycle.

The inventors have performed a thermodynamic simulation to model different possible configurations of the SCWG system of the present invention enabling fuel production and power generation cycles. The simulation was built using a UniSim Design plant and process simulation software (Honeywell). A complete stoichiometric conversion of the inlet organic content, and the required amount of additional water, into products including H₂, CH₄, and CO₂ was assumed.

For feed material composed of glucose in water, the reaction balance is:

C₆H₁₂O₆+5H₂O [symbol] 10H₂+0.5 CH₄+5.5 CO₂ (4)

The mixture entering the reactor includes also excess water and a small fraction of inorganic material found in the feed organic matter. The needed reaction heat, to be supplied from an external source, is calculated based on the difference in enthalpies of the inlet and outlet streams.

The simulation was used to define a cogeneration SCWG plant producing both fuel and power. Two separate reference plants (not shown in the figure) for production of fuel-only and power-only were defined, such that the total input of raw feedstock and thermal energy the two separate plants is equal to the input into the cogeneration plant. The output of fuel was converted to an equivalent amount of electricity assuming the use of a fuel cell, so that an overall effective efficiency can be defined. The biomass input is evaluated as equivalent thermal energy, using the heating value of the organic material. These definitions allow calculation of an equivalent efficiency for comparison of the cogeneration approach to the production of power and fuel using separate reference plants. The overall plant efficiency for the cogeneration cycle was analyzed, in comparison to the combined efficiency of a fuel production and power generation plants.

The comparison is performed with variations in five important design parameters: (1) reactor temperature (also turbine inlet temperature): 400-600° C.; (2) turbine expansion stages with intermediate reheat: 1, 2, 3; (3) fuel cell efficiency (relative to H₂ LHV): 50-70%; (4) turbine isentropic efficiency: 75-95% and (5) biomass feedstock mass fraction: 5-25% (the rest is water).

The criterion used for optimization and cycle comparison is the cycle efficiency. The energy input into the cycle \dot{Q}ₜₒₜ is measured in heat-equivalent units, including both the externally supplied heat and the caloric value (LHV) of the feed material:

\dot{Q}ₜₒₜ=ΣQ^{&}ⱼ+Σm^{&}ⱼhⱼ (5)

where \dot{m}ⱼ and hⱼ are the mass flow rates and specific enthalpy (including enthalpy of formation) of all matter streams into and out of the cycle. Qⱼ are the heat transfer rates for all heat inputs to the cycle from the surroundings.

The energy output from the cycle (\dot{W}_{dot}) is measured in electricity-equivalent units. Work is converted to electricity with efficiency of 95%. Work inputs (e.g., pump) are subtracted from work output to produce net output of work. The output of hydrogen fuel is converted to work-equivalent units by assuming that the fuel produces electricity in a fuel cell of given efficiency. The fuel cell efficiency is the ratio of its electricity output to the caloric value (LHV) of the fuel input.

The overall cycle efficiency (first-law efficiency) is the ratio of equivalent work output to equivalent heat input:

η₁=W^{&}ₜₒₜ/Q^{&}ₜₒₜ (6)

The small amount of methane also produced usually cannot be converted in the same fuel cell as the hydrogen. Therefore, the methane may be recycled to provide a part of the reactor heating.

It should be noted that the cogeneration cycle requires a high fraction of organic matter in the input mixture. If the organic fraction is too low, the trend is reversed and the combined cycle produces less work than separate cycles. This was repeatedly observed in simulation results. This effect is the result of the restriction on the expansion pressure: in the fuel production and cogeneration cycles, the expansion is stopped at 7.5 bars in order to allow H₂ separation by PSA. In the pure power production cycle, the expansion is allowed to continue, reaching a much lower pressures, and producing more output work. The combination of these two effects produces this cross-over result, where a minimum amount of organic matter is needed in order to realize the advantage of the cogeneration cycle.

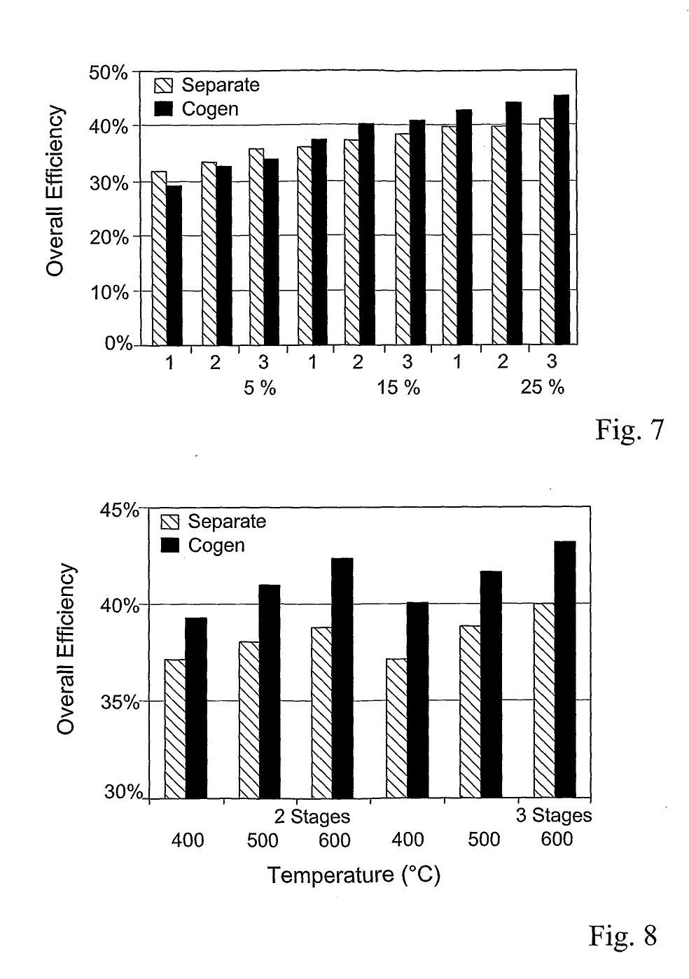

Reference is made to FIG. 7 illustrating the overall efficiency behavior at 600° C. for different feedstock mass fractions (5% biomass, 15% biomass, 25% biomass) and different number of turbine expansion stages. The overall efficiency includes both the cogeneration cycle and the sum of the separate reference cycles, while changing the feedstock mass fraction and the number of turbine expansion stages at a fixed temperature of 600° C. The results show conversion efficiency from heat to electricity of up to 45% for the cogeneration cycle under moderate assumptions for the performance of the system components (isentropic efficiencies of the turbine, and conversion from hydrogen to electricity). Under more optimistic assumptions (high isentropic efficiencies and high conversion of the hydrogen to electricity) the cogeneration cycle efficiency is more than 50%. These results are very high, competitive as compared to the usual range of combined cycles (gas and steam turbines), which require much higher operation temperature of above 1,300° C. The results predicted for the cogeneration SCWG cycle require a temperature of only 600° C.

The synergy between the chemical and thermodynamic components in the cogeneration cycle can be clearly seen in most cases in FIG. 7. The cogeneration cycle has a clear advantage over the superposition of the two references cycles. This efficiency advantage is clear for two and three turbine expansion stages.

Therefore, the cogeneration of fuel and electricity has been compared with the production of fuel and generation of electricity in two separate cycles. The experimental results show that the cogeneration cycle exploits the potential of the product mixture for producing work by expansion, and can reach higher second-law efficiency of up to 59%. This result indicates a good exploitation of the thermodynamic potential of the resources (biomass and heat), which cannot be achieved with separate process for fuel production and power generation. This is a significant advantage relative to the separate operation of the fuel production and power production cycles, validating the benefit and synergy of the cogeneration approach.

Reference is made to FIG. 8 illustrating the overall efficiency behavior at fixed feedstock mass fraction for different temperatures and different number of turbine expansion stages. In both parameters, increasing the number of expansion stages and increasing the temperature improve the efficiency, at the cost of additional complexity and cost of materials. This implies that optimization of the cycle performance has to take into account the different process parameters rather than optimizing each parameter separately.

Another element influencing the cycle performance is the ability to recover the energy contained in the exit stream—the mixture leaving the reactor (in chemistry only cycle) or the turbine (in the power and cogeneration cycles). The power only cycle cannot offer any heat recovery, since the expansion in the turbine is all the way to near ambient temperature. In the cogeneration cycle, the to mixture leaving the turbine is still at elevated temperature and pressure (7.5 bar, due to the requirement of the separator), and therefore it can be heat recovered. The inventors have calculated that about 25-45% of the available energy can be recovered for preheating of the inlet feed into the reactor. The recovered fraction increases with the number of expansion stages, since more expansion stages with reheat produce higher quality and higher enthalpy of the available steam at the exit of the turbine.

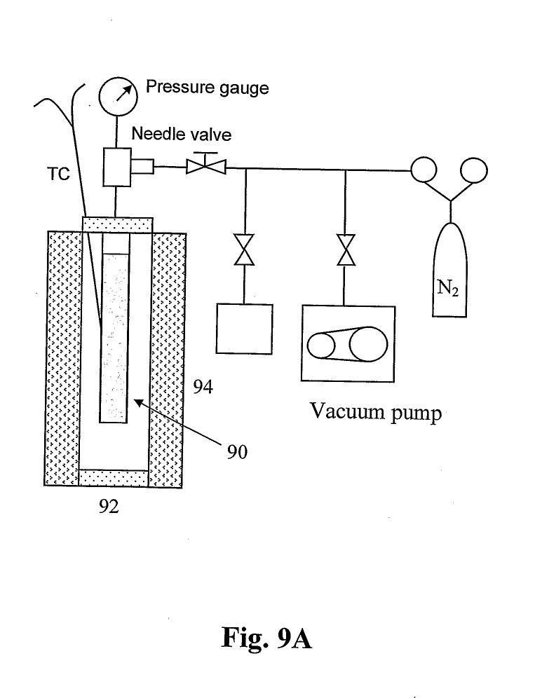

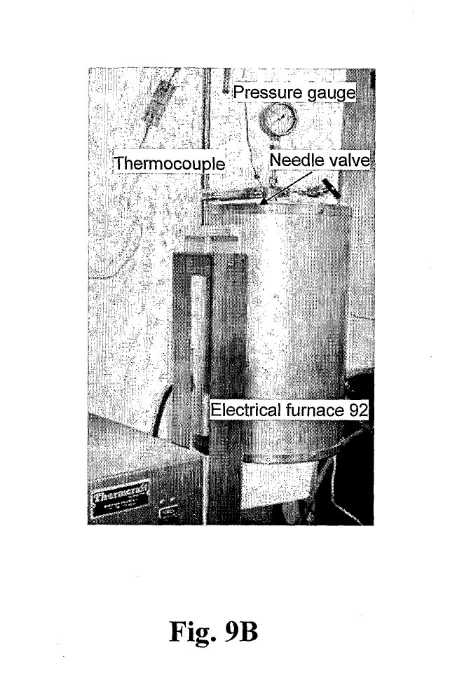

Reference is made to FIGS. 9A-9B illustrating a schematic representation and experimental implementation of an example of the reactor of the present invention.

Two batch reactor systems have been developed for the gasification of different kinds of organic feedstock under supercritical water conditions, with and without a catalyst system. The process includes heating of the reactor system loaded with the feedstock, holding the temperature for a specified residence time, and cooling the reactor when the reaction is terminated.

A batch micro-reactor (volume=1.4 ml) for SCWG has been built, allowing flexible operation and fast performance of short experimental runs. Three tubes are fixed into the top cover. One tube is for the pressure transducer and for a thermocouple. Two tubes serve for input and exit of inert gas and also for the exit of the products of reaction. The reactor has an additional outlet equipped with a safety relief valve. The reactor is configured to maintain pressure of up to 35 MPa and temperature of up to 550° C.

In this specific and non-limiting example, the SCWG pressure reactor system is a batch micro reactor 90, surrounded by a controllable electric furnace 92 (e.g. Thermcraft with controller), a pressure gauge, a needle valve and a sampling bag 94 for gases.

A larger lab reactor, which has internal volume of 75 ml, was also designed and fabricated. This reactor is capable of holding internal pressure of up to 500 bars and at temperatures of 450° C.

The experiment starts by loading the mixture of organic feed material and water into the reactor and sealing the reactor. Several organic substances were used: glucose, cellulose, rice husks, and wheat stalks. These feedstocks were mixed with water and the catalyst and inserted into the cleared reactor. In preparing the reactor for the experiment, the air was replaced by helium at initial pressure of about 100 bars. The reactor is placed in a dry ice bath to freeze the solution, so that the solution does not evaporate during the evacuation of the air inside the reactor. After pumping the air out of the system, N₂ is introduced to achieve an initial pressure of 10-12 MPa in order to avoid the vaporization of water during the heating process and condensation on colder parts, e.g. the pressure gauge, thus, causing a difficulty to achieve the critical conditions of the water. The reactor is heated to the desired temperature and pressure, i.e. the reactor was placed in the furnace and heated up to 550° C. The pressure increased in the reactor up to 350 bars. During the operation, the temperature was measured by the thermocouple attached to the outer wall of a reactor. When the process steady-state conditions have been reached, the reactor was retained in the furnace for a certain residence time and then it was rapidly pulled out from the furnace and cooled down in air to room temperature. The pressure in the system depends on the reaction temperature and the amount of the produced gas.

Small samples of the gaseous products were extracted at specific intervals of residence time under the conditions of supercritical water. The high-pressure samples are adiabatically expanded to a relatively large volume, which enables fast and irreversible cooling of the gas without change in its composition. At the end of the test, the reactor is cooled fast to room temperature. The products are released from the reactor through a pressure regulator. These products are collected in a bag when the reactor reached room temperature that was evacuated prior to the test with a vacuum pump. The gaseous products were analyzed by a gas chromatograph (GC).

The experiments were carried out with 0.25-1.0M glucose solution in water, at temperature of 450° C. Preliminary results show that the conversion yields can be high, up to 98%, and the proportion of hydrogen can reach over 50% in the gas phase. The influence of residence time on the gasification efficiency was preliminary tested and it was shown that at least 60 seconds are required as minimum residence time under these conditions. The carbon conversion percentage X_{C} is an indicator of the extent of the SCWG process and is defined as the degree of conversion of carbon from biomass to final gas phase:

$${X}_{C} = \frac{{\sum }_{i} {N}_{C , product \_ gas , i}}{{N}_{C , feed}} \times 100 \qquad ( 7 )$$

N_{C,product}_{_{—}}_{gas,i} is the number of carbon moles in the product gas i, and N_{C,feed} is the number of moles of carbon in the feedstock.

Table 1 shows calculation results of an example experiment of supercritical water gasification of glucose. The feed material used in this experiment was 1.5 ml of 0.25 molar glucose solution, and 0.07 g K₂CO₃ as a catalyst. The experimental conditions were pressure of 220 bars and temperature of 530° C. The table shows the concentration of the product gases in volume percentage both in a mixture with helium, which was used as the carrier gas (generally the content of the product gases were about 10% in helium), and in the separated product mixture. The carbon conversion in this experiment was X_{C}=83.8%.

TABLE 1
Example of the analysis results of supercritical water gasification of Glucose

| | Chromatogram SCWG of 1.5 ml Glucose Solution (3.8 g H2O; 0.35 g Glucose; | | | | | | | |
|---|---|---|---|---|---|---|---|---|
| | 0.07 g K2CO3). Reactor temperature 530° C.; Pressure in the reactor 220 Bar. | | | | | | | |
| | | | | | | % vol | | |
| | | | | | %, in | %, in gas | P_{gas} | |
| Peak | D(296K) | RT | AREA | K | mix | pyrolysis | pyr, g | B * F/100 |
| H2 | 0.0829145 | 2.588 | 2473 | 813 | 3.04 | 29.23 | 0.004 | 0.00 |
| air | 1.1925304 | 2.895 | 36133 | 46958 | 0.77 | 7.39 | 0.013 | 0.01 |
| CO | 1.1528716 | 3.01 | 34649 | 40713 | 0.85 | 8.18 | 0.014 | 0.01 |
| CH4 | 0.6594426 | 3.71 | 54032 | 35315 | 1.53 | 14.70 | 0.014 | 0.01 |
| CO2 | 1.8113919 | 6.256 | 201312 | 48253 | 4.17 | 40.08 | 0.107 | 0.08 |
| C2H4 | 1.1537939 | 8.025 | 1976 | 48268 | 0.04 | 0.39 | 0.001 | 0.00 |
| C2H6 | 1.237723 | 9.417 | 0 | 43481 | 0.00 | 0.00 | 0.000 | 0.00 |
| C2H2 | 1.0717095 | 11.041 | 0 | 48063 | 0.00 | 0.00 | 0.000 | 0.00 |
| | | | | | 10.41 | 99.97 | 0.152 | 0.11 |
| He | 0.1643 | | | | 89.59 | | | 0.15 |

| | | | | | | P_{gas} |
|---|---|---|---|---|---|---|
| glucose, g | P_{gas} + P_{He} | V_{gas} + VHe | V_{gas}, L | VHe, L | P_{He}, g | pyr, g |
| 0.138 | 0.36 | 1.412616 | 0.15 | 1.27 | 0.20794 | 0.139 |

| N_{c feed} | N_{c product-gas} | X_{c}, % |
|---|---|---|
| 0.0046 | 0.003856 | 83.8 |

Data obtained on the basis of such analyses for experiments with different feedstock substances and a K₂CO₃ catalyst is summarized in the Table 2. The fraction of free hydrogen was in the range of 30-40% of the product mixture. The fraction of methane was 11-15%, and the fraction of higher hydrocarbons was very low in all cases.

TABLE 2
Result of SCWG experiments with different feed materials and
with K₂CO₃ as a catalyst.

| | |
|---|---|
| % Carbon | Gas composition from SCWG, % volume |

| Material | conversion | H₂ | CH₄ | CO₂ | CO | C₂H₆ |
|---|---|---|---|---|---|---|
| Glucose | 84 | 29.23 | 14.7 | 40.08 | 8.18 | 0 |
| Rice husk | 44.7 | 37.76 | 11.2 | 36.27 | 0 | 1.91 |
| Wheat | 85.39 | 37.08 | 13.32 | 34.61 | 0 | 2.03 |

The results for cellulose with K₂CO₃ catalyst are shown in Table 3.

The reaction equation of the gasification of cellulose in supercritical water is as follows:

C₆H₁₀O₅+7H₂O [symbol] 12H₂+6CO₂ (8)

The results show that the content of H₂ in gas composition is more than twice that without the catalyst. The H₂ yield is 2.7 mole/kg of cellulose without catalysts and about 10.0 mole/kg with catalysts.

TABLE 3
Result of the influence of the catalyst K₂CO₃ on the SCWG of
Cellulose at the temperature of about 550° C. and pressure 350 bar.

| | | Product gas composition from | | | | |
|---|---|---|---|---|---|---|
| | % Carbon | SCWG, % volume | | | | |
| Catalyst | conversion | H₂ | CH₄ | CO₂ | CO | C₂H₂ |
| Yes | 39.6 | 37.0 | 13.6 | 38.65 | 0 | 3.49 |
| No | 25.16 | 14.79 | 16.35 | 52.18 | 7.36 | 3.65 |

The experimental results from the batch reactors have shown that the SCWG can be effective for a range of feedstock materials.

1. A system for producing a high pressure product gas under super-critical water conditions; said system comprising:
a pressure reactor accommodating a feed mixture of water and organic matter;
a solar radiation concentrating system heating the pressure reactor and elevating the temperature and the pressure of the mixture to about the water critical temperature point and pressure point or higher;
said reactor being configured and operable to enable a supercritical water process of the mixture to occur therein for conversion of said organic matter and producing a high-pressure product fuel gas.

2. The system of claim 1, comprising a pump for pumping said feed mixture, the pump being operable to elevate the pressure of the mixture to be about the water critical pressure point or higher.

3. The system of claim 1, wherein said pressure reactor is a solar reactor directly heated by said solar radiation concentrating system.

4. The system of claim 1, wherein said pressure reactor is indirectly heated by said solar radiation concentrating system.

5. The system of claim 1, comprising a turbine connected to the pressure reactor for receiving the high-pressure product gas; and an electrical generator connected to the turbine, enabling simultaneous cogeneration of electrical power.

6. The system of claim 1, comprising one or more separators for receiving the high-pressure product gas and separating at least one of water, H₂ and CO₂.

7. The system of claim 3, wherein said solar radiation concentrating system comprises a solar collector directly elevating the temperature of the mixture to about 400° C. or higher.

8. The system of claim 4, wherein said solar radiation concentrating system comprises a solar collector indirectly elevating the temperature of the mixture to temperatures of about 400° C. or higher by using a heat transfer fluid circuit.

9. The system of claim 1, wherein said solar collector is selected from parabolic trough collector, solar tower, and solar tower comprising a tower reflector.

10. The system of claim 1, comprising a thermal storage unit configured and operable for continuously operating said pressure reactor regardless to variations of solar energy.

11. The system of claim 1, comprising a heat exchanger configured and operable to use thermal energy of said high-pressure product gas to elevate the temperature of the mixture.

12. A system for producing a high pressure product gas under super-critical water conditions; said system comprising:
a pressure reactor accommodating a feed mixture of water and organic matter;
a heating system heating the pressure reactor and elevating the temperature and the pressure of the feed mixture to about the water critical temperature point and pressure point or higher;
the reactor being configured and operable to enable a supercritical water process of the mixture to occur therein for producing a high-pressure gas;
a turbine connected to the pressure reactor for receiving the product gas; and;
an electrical generator connected to the turbine, enabling cogeneration of electrical power.

13. The system of claim 12, comprising a pump for pumping the feed mixture into said pressure reactor, the pump being operable to elevate the pressure of the mixture to be about the water critical pressure.

14. A catalyst system comprising at least one metal and an oxide support, said oxide support comprising at least one of Al₂O₃, MnₓO_{y}, MgO, ZrO₂, and La₂O₃, or any mixtures thereof; said catalyst being suitable for catalyzing at least one reaction under supercritical water conditions.

15. The catalyst system of claim 14, wherein said metal is selected from ruthenium, rhodium, nickel or any mixtures thereof.

16. The catalyst system of claim 15, wherein said metal is ruthenium.

17. The catalyst system of claim 15, wherein said metal comprises at least about 98 weight percent of ruthenium.

18. The catalyst system of claim 15, wherein said catalyst system comprises alkali salts including at least one of K₂CO₃, KOH, NaOH, Ca(OH)₂ and Na₂CO₃ or any mixtures thereof.

19. The catalyst system of claim 14, comprising 1 wt. % to 5 wt. % of ruthenium.

20. The catalyst system of claim 19, comprising 1 wt. % to 2% wt. % of ruthenium.

21. The catalyst system of claim 14, wherein said oxide support comprises MnₓO_{y} in a concentration not exceeding 10 wt. %, MgO in a concentration not exceeding 10 wt. %, La₂O₃ in a concentration not exceeding 10 wt. % and α-alumina.

22. The catalyst system of claim 14, wherein said at least one reaction is selected from supercritical water gasification and decomposition of organic compounds in aqueous phase.

23. A method for providing a product gas, said method comprising: providing a reactant mixture containing water and organic matter; providing a catalyst system of claim 14; reacting said reactant mixture in presence of said catalyst system under supercritical water conditions; thereby obtaining a product gas.

24. The method of claim 23, wherein said supercritical conditions comprises temperature of about 374° C. or higher and pressure of about 220 bars or higher.

25. The method of claim 23, wherein said organic matter comprises low-quality organic residues and waste such as residues from fermentation, anaerobic digestion, biomass, organic waste with high moisture content, agricultural and forestry waste; bagasse or other organic waste from food processing industry; waste from bioethanol or biodiesel production processes; organic sludge from water treatment plants and refineries, marine algae, algal biomass sludge, sewage sludge or algae broth, biomass polymers comprising cellulose, hemicellulose and lignin.

26. The method of claim 23, wherein said product gas comprises high-quality gas containing hydrogen, carbon dioxide, methane and CO.

27. The method of claim 23, comprising separating at least one of water, H₂ and CO₂ from said product gas.

28. The method of claim 23, comprising sequestrating CO₂ from said product gas.

29. The method of claim 23, comprising processing said product gas to produce liquid fuel.

30. The method of claim 23, comprising: pumping said reactant mixture into a pressure reactor, the pressure of the mixture being elevated to about the water critical pressure point; heating said pressure reactor to elevate the temperature of said pressurized mixture to about the water critical temperature point or higher; interacting between said reactant mixture and said catalyst system inside said pressure reactor to enable a super critical water process to occur within said pressure reactor such that high pressure product gas is produced.

31. The method of claim 23, comprising powering a turbine with said product gas; and powering an electrical generator with said turbine.

32. The method of claim 23, comprising: heating said pressure reactor to elevate the temperature and the pressure of the mixture to about the water critical temperature and pressure point or higher; interacting between said reactant mixture and said catalyst system inside said pressure reactor to enable a super critical water process to occur within said pressure reactor such that high pressure product gas is produced; powering a turbine with said product gas; and powering an electrical generator with said turbine.

33. The method of claim 31, comprising using thermal energy of said high-pressure product gas to elevate the temperature of said mixture.

34. The method of claim 31, wherein heating said pressure reactor comprises using a solar energy source.

35. The method of claim 34, comprising storing heat produced by said solar energy source to enable continuous gasification.
